(19) United States
(12) Patent Application Publication
(10) Pub. No.: US 2008/0200586 A1
(43) Pub. Date: Aug. 21, 2008

(54) Dental/Surgical Sealants Including Shapeable Particles

(76) Inventors: Paul Stephen Holden, Sheffield (GB); Alexandra Elizabeth Heath, Sheffield (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/632,104

(22) PCT Filed: Jul. 18, 2005
(86) PCT No.: PCT/GB2005/002811

§ 371 Date: Sep. 27, 2007

(30) Foreign Application Priority Data
Jul. 16, 2004 (GB) 0415981.0

(51) Int. Cl. A61L 24/04

(52) U.S. Cl. 523/117; 523/116

(57) ABSTRACT

A sealant composition, suitable for surgical or dental use, comprises a sealant and, dispersed therein, one or more particles of a material which is prestressed and/or capable of undergoing expansion or contraction.

### FIELD OF THE INVENTION

This invention relates to a sealant composition, suitable for surgical or dental use.

### BACKGROUND TO THE INVENTION

Surgical and dental sealants generally consist of a polymerisable binder, reinforced with inert organic or inorganic filler particles. Also known are glass ionomer cements. The cements, however, have limited physical strength, making them unsuitable for surfaces that undergo high physical stress.

Dental sealants are often used in conjunction with dental insert plugs. The plug is inserted into a cavity and the sealant used to fill the gaps that remain between the walls of the cavity and the plug.

A common problem with dental sealants is that they do not provide adequate bonding strength at the sealant/dentine interface, leading to peeling and bacterial penetration. Conventional sealants are generally hydrophobic, making for a poor interface with dental tissues, which are principally hydrophilic. Further, the nature of some sealants is such that it is often difficult to remove all air pockets and bubbles from the root canal, thereby increasing the likelihood of infection. Even in the unlikely event that the seal is initially perfect, shrinkage or hydrolytic degradation normally occurs over time, causing voids or fissures.

In order to address these shortfalls, numerous solutions have been proposed. Examples include etching the surface of the dentine with acid or an acidic primer, e.g. a methacrylate derivative of a carboxylic or phosphoric acid. Primers, however, need to be subsequently washed from the tooth, resulting in longer treatment times. Also, the acidic monomer of the primer can often remain on the tooth surface after washing, resulting in poor adhesion of the sealant to the tooth surface.

Further, it has recently been appreciated that root canals are not always of circular cross-section; some are of a substantially oval cross-section. Since insert plugs are generally of circular cross-section, a dental sealant must be used to fill the remaining space. While this may not sound problematic in theory, obtaining a satisfactory seal has proven difficult in practice. Similar difficulties are experienced when the dental cavity has not been properly prepared.

Analogous problems are encountered with surgical sealants. For example, the application of bone cement can result in the formation of voids at the bone interface. This may reduce or inhibit mechanical function and allow bacterial ingress.

### SUMMARY OF THE INVENTION

It has been discovered that many of the limitations described above can be overcome, according to the present invention, by dispersing, within a liquid sealant, particles of a material which is prestressed and/or capable of expansion or contraction.

The invention provides compositions that can swell, contract or change shape, thereby ensuring a complete seal, without the need for priming or etching. For example, a sealant of the invention may be used in dental root filling, where the volume change of the sealant may be sufficient to eliminate air voids, even in an irregular root canal cavity.

### DESCRIPTION OF THE PREFERRED EMBODIMENTS

The term “sealant composition” as used herein refers to compositions suitable for use as, for example, sealants, adhesives, restorative (filling) materials, cements, implant components, bone substitutes and the like.

The degree of expansion, contraction or shape change may depend on the nature and/or components of the sealant, the intended application, and the precision and expertise of the practitioner. For a composition which expands, it is preferred that the swelling is in the range of about 0.5% to about 150%, more preferably from about 2 to about 100%, and most preferably from about 10 to about 100%, by volume.

The particles may be in any suitable form, for example as spheres, fibres, plates and the like. The particles may be obtained using any suitable method known in the art. Preferred techniques include grinding and emulsion polymerisation.

The particulate material is preferably prestressed, allowing its shape to be relaxed, for example, by the action of heat and/or hydration. The purpose of prestressing is to change the initial shape so as to arrive at some other desired shape upon hydration or heating, i.e. to effect a change of shape. The prestressed material may or may not also undergo expansion or contraction when hydrated. Upon heating and/or hydration, the material becomes less rigid and the shape change imposed by prestressing relaxes. Prestressing may be effected using any suitable method known in the art; see, for example, the techniques described in GB-A-2139898 and GB-A-2340430.

The particles may be able to expand/contract and undergo a change in shape. In particular, the particulate material may be prestressed so that it undergoes anisotropic expansion. This may be achieved by the application of stress to the dry material, to give a particle having a controlled aspect ratio. The particles can then be oriented in the sealant using a suitable flow-controlled process. When heated or hydrated, the composition can then expand anisotropically, by virtue of the particles' both expanding and recovering their original shape. This type of expansion is desirable since it may allow for the filling of lateral cavities in a tooth cavity.

The particulate material may be a hydrophilic material. In this case, the equilibrium water uptake of the material is preferably from about 10 to about 99%.

The particles are preferably made of a polymeric material. Suitable materials include addition polymers or copolymers of monoethylenically unsaturated monomers. If the polymer is required to be water-swellable, then hydrophilic monomers can be used. Suitable monomers include hydroxymethyl methacrylate, N-vinyl-2-pyrrolidone (VP and other vinyl lactams, acrylamide, methacrylamide and N-substituted derivatives thereof. Substituted acrylamide and methacrylamide derivatives may be mono- or di-substituted, preferred substituents including alkyl, hydroxylalkyl and aminoalkyl (including mono- and di-substituted aminoalkyl).

The particulate material may be a copolymer, preferably one obtained by copolymerisation of a hydrophilic monomer with an alkyl acrylate. Examples of suitable copolymers include copolymers of VP and methyl methacrylate (MMA); VP and hydroxymethyl methacrylate; and VP, styrene and acrylonitrile; and VP and terephthalic acid.

It is also preferred that the particulate material is cross-linked. This may be achieved by incorporating di- or poly-functional cross-linking agents within the monomer system. Examples of suitable cross-linking agents include allyl methacrylate, divinylbenzene, ethylene glycol dimethacrylate, trimethylol propane trimethacrylate and the like. Light cross-linking is preferred, the cross-linking agent being used, for example, in an amount of about 1% by weight of the monomer system. For a high-strength polymeric material, cross-linked copolymers of acrylonitrile or methacrylonitrile and VP are suitable.

The particulate material may be thermolabile, allowing for a temperature-dependent transition. In this case, the material preferably undergoes a shape change around body temperature. Materials that undergo such transitions are known, for example, in the intraocular lens industry. Suitable materials include those described in EP-A-0308130, EP-A-0269288, WO94/07686 and EP-A-0766952, the content of each of which is incorporated herein by reference.

The composition may comprise a radio-opaque material. When the particulate material is polymeric, then it is preferred that it contains a non-leachable radio-opaque species. The non-leachable radio-opaque species can be covalently attached to the polymer or to a monomer, or suspended in particulate form. Alternatively, the radio-opaque species may be present in the liquid sealant.

The radio-opaque species may be a halogenated aromatic compound, substituted with a reactive functional group. Examples of such compounds include substituted aromatic triiodides, tribromides, trifluorides and trichlorides. The reactive functional group may be a hydroxyl, carbonyl, amide, amine, carbonyl, thiol, allyl, vinyl or anhydride group. Further examples of reactive functional groups include isocyanates, esters, aldehydes, N-hydroxysuccinimide esters, epoxides, carboxylic esters, tresylates, alkyl halides, carboxylic acids, haloketones, alkenes, alkynes or acyl chlorides.

Further examples of radio-opaque species that can be used include triiodobenzoyloxythyl methacrylate, amidotrizoate, iothalamate, iohexyl, iopamidol, iopromide, ioxaglic acid, iopadate, iotroxate, iobenguane, iobenzamic acid, iocarmic acid, iocetamic acid, iodamide, iodipamide, iodixanol, iodised oil, iodoalphionic icid, p-iodoaniline, o-iodobenzoic acid, iodochlorohydroxyquin, o-iodohippurate sodium, o-iodophenol, p-iodophenol, iodophthalein sodium, iodopsin, iodopyracet, iodopyrrole, iodoquinol, iofetamine I, ioglycamic acid, iohexyl, iomeglamic acid, iopamidol, iopanoic acid, iopentol, iophendylate, iophenoxic acid, iopromide, iopronic acid, iopydol, iopydone, iothalamic acid, iotrolan, ioversol, ioxaglic acid, ioxilan and ipodate.

The radio-opaque materials may be provided for use in the form of a suspension (e.g. within a polymeric material). Such materials include tungsten carbide, barium sulphate, zirconium oxide and bismuth iodide.

Mixtures of such materials may be used. Other pigments or fillers may also be present, for coloration or to improve handling characteristics of the composition.

Any suitable surgical or dental sealant known in the art may be used, a preferred sealant being AH Plus™, obtainable from Dentsply. Other polymeric, curable resins are also preferred. Alternatively, the sealant may be a glass ionomer cement; the presence of the particulate material counteracts the tendency of such materials to shrink in use.

It will be appreciated that the viscosity (and other characteristics) of the liquid sealant is likely to be dependent upon the intended application of the composition. The sealant may be in the form of a paste or cement, or in a relatively less viscous form. The required viscosity will be apparent to those skilled in the art. The sealant should retain adequate elasticity to permit the desired change in shape and/or volume of the particles.

For hydrophilic particles, the liquid sealant should permit the ingress of water to induce the swelling of the particles. For high particle loadings, e.g. >45%, it is likely that water can simply permeate through one particle to the next. For lower loadings, the particles may not be in contact with one another, and so it is preferred that the liquid sealant is water-permeable. Also, since handling behaviour can be adversely impacted by high particle loading, it is generally preferred that the liquid sealant has some intrinsic water-permeability. Preferred sealants in this respect include polymer systems which are based on cyanoacrylate, epoxyamine or methacrylate monomers. The polymer system may be derivatised with alkyl methacrylate, alkylamino methacrylate or oxyphosphoro methacrylate groups.

In an embodiment of the invention, the composition comprises an active agent, in the sealant and/or the particles. The agent may have anti-microbial analgesic, anti-inflammatory or anaesthetic activity. If anaesthetic, the composition may then have the potential to reduce pain and trauma associated with the treatment, when the anaesthetic administered to undertake the treatment wears off. If anti-microbial, the composition can reduce or inhibit the infection which may arise significantly after the treatment. This is largely due to bacterial ingress and colonisation of the root canal as a result of the breakdown and partial dissolution of the sealant, causing a void in the obturation.

The term “agent” is used herein to describe one or more active materials. More than one active ingredient may be used, for example, an analgesic for pain relief and an anti-inflammatory to reduce swelling. Any one or more active ingredients may be combined with an antimicrobial, to prevent infection.

The release of an agent is dependent on a range of factors, including solubility of the active in body fluid, volume of fluid available, hydrophilic polymer properties, loading in polymer, presence of excipients and geometry and physical form. The effectiveness of the agent will also depend on its potency.

The solubility of the active is a function of the nature of the active and the body fluid. By way of example, various agents may be formulated with salts having different solubilities in water or body fluids. The choice of agent and salt forms can be used to influence the release rate.

The volume of fluid available depends on the application under consideration. For application in dental root canal treatment, the sealant is used to fill a prepared tooth cavity which is then capped. Effectively the sealant is enclosed and the free volume available for expansion of the sealant is restricted. This means that the volume of the fluid available to swell the hydrophilic polymer is limited.

The release of the active may be controlled by saturation of the body fluid in the cavity. The level of the agent will be dependent on various factors such as the solubility, the elimination rate (i.e. how quickly the active is broken down or removed), the loading in the polymer, the release rate from the polymer, diffusion through the resin and the properties of the hydrophilic polymer.

If the release from the polymer is rapid and the removal of the active is slow, then active levels are likely to be controlled by the solubility in the fluid. If the volume of fluid is high, then the release is more likely to be controlled by the properties of the polymer and sealant.

The basic release mechanism of the active is the dissolution of the active and diffusion out of the hydrophilic polymer. Hence the release characteristics may be changed by controlling the loading of the active in the polymer, the amount of fluid in the hydrophilic polymer (swelling of the polymer) and the diffusion out of the polymer.

Swelling of the polymer can be controlled by a number of means. For example, formulation of the polymer can be varied to control the hydrophilicity, which will affect the solubility of the body fluid in the polymer. The degree of cross-linking introduced can be used to limit the amount of swelling possible in the polymer, with more heavily cross-linked materials having lower swelling.

Depending on the diffusion kinetics, release of the active can be determined by either the migration of the body fluid into the polymer or the diffusion of the agent out of the swollen polymer. Generally, lowering the water uptake of the material reduces the release rate of the active.

Excipients can be introduced to modify either the solubility of the agent in the body fluid or to increase the swelling of the hydrophilic polymer. An example is the inclusion of glycerol (or similar polyols such as polyethylene glycol) in the hydrophilic polymer. Glycerol significantly increases the swelling of the polymer.

Cyclodextrins may be used to absorb the active material. In many cases, the use of cyclodextrin enhances the solubility of sparingly soluble materials. For example, the water-solubility of ibuprofen may be enhanced when used in conjunction with beta-cyclodextrin.

The release characteristics of an active dissolved or dispersed in hydrophilic polymer are influenced by geometry, as the body fluid has to diffuse in to the hydrophilic polymer and the active has to diffuse out of the swollen polymer. The time to diffuse through a material depends significantly on thickness. Hence as the thickness of the polymer increases, the path length increases (fluid and active have further to diffuse) and the release rate is reduced.

The nature of the active agent is not critical. It can be any soluble material with a pharmacological effect, either local or systemic. Preferred agents include antimicrobial compounds such as chlorhexidine digluconate, chlorhexidine acetate, chlorhexidine hydrochloride, triclosan, povidone iodine, silver nitrate, silver sulphadiazine, metronidazole and nitrofurazone. Further compounds that can be used include ammonium hydroxide (a compound used to clean root canal cavities prior to filling), anti-inflammatory agents such as ibuprofen, analgesics such as aspirin, anaesthetics such as lignocaine (chloride), amethocaine and bupivacaine (chloride), and other supplements such as vitamins and sodium fluoride. Some agents may require solubility enhancement, e.g. by the use of cyclodextrin complexing.

The following Examples illustrate the invention. All percentages and proportions are by weight/volume unless otherwise stated.

### Example 1

An epoxy-amine-based sealant, AH Plus™, was obtained from Dentsply. Equal volumes of its two paste components were mixed with an equal volume of particles of a copolymer of MMA and VP. The equilibrium water uptake of the material was about 75%. A smooth paste was formed, which was then shaped into rods 10 mm long and approximately 3 mm in diameter. The rods were hydrated in distilled water. After about 8 hours of hydration, the sealant composition exhibited an increase in volume of approximately 113%.

The sealant composition of Example 1 was introduced into prepared teeth with a size 40 Gutta Percha dental point. The teeth were hydrated in water for 3 days, and then placed in a water-based dye. As a control, the experiment was repeated using AH Plus sealant. The penetration of the dye was found to be significantly reduced for the teeth which were treated using the composition of the invention.

A sectioned tooth prepared as described in above was placed in a water-based dye for 7 days. Imaging showed that the sealant filled the lateral canal and prevented dye ingress.

The paste was placed in a dental training system (for training for preparing cavities). The system was photographed, then the sealant was hydrated for 1 week. The sealant could be seen to have hydrated and expanded in the arms of the training system.

### Example 2

A sealant composition was prepared according to Example 1, and mixed with an equal volume of radio-opaque poly(hydroxyethyl methacrylate) spheres, prepared according to Horak et al, Biomaterials (1998) 19: 1303-1307. The radio-opacity of the sealant composition was then assessed using conventional X-ray equipment. The composition was confirmed as being radio-opaque.

### Example 3

A 2-component amine-epoxy resin supplied by Bostik Findley Limited (Araldite Precision) was mixed with approximately 25% by weight of a MMA/VP cross-linked co-polymer as described in Example 1. The resultant paste was shaped into 10 mm long rods, as described in Example 1. Immediately after moulding, the rods were hydrated in distilled water. After 24 hours' hydration, the sealant composition exhibited a weight increase of approximately 31%. After 48 hours' hydration, the weight increase was approximately 36%, which rose to 39% after 144 hours.

### Example 4

A single-component cyanoacrylate resin supplied by Loctite Limited (SuperGlue Gel) was mixed with approximately 25% by weight of a MMA/VP cross-linked co-polymer as described in Example 1. The resultant paste was shaped into 10 mm long rods, as described in Example 1. Immediately after moulding, the rods were hydrated in distilled water. After 24 hours' hydration, the sealant composition exhibited a weight increase of approximately 9%. After 48 hours' hydration, the weight increase was also 9%, but rose to 12% after 144 hours.

### Example 5

A single-component cyanoacrylate resin supplied by Bostik Findley Limited (SuperGlue Precision) was mixed with approximately 50% by weight of a MMA/VP cross-linked co-polymer as described in Example 1. The resultant paste was shaped into 10 mm long rods, as described in Example 1. Immediately after moulding, the rods were hydrated in distilled water. After 24 hours' hydration, the sealant composition had a weight increase of approximately 15%. After 48 hours' hydration, the weight increase was 17%, which rose to 21% after 144 hours.

### Example 6

A 2-component zinc oxide-Eugenol-based root canal sealer supplied by Kerr Corporation (Tubli-Seal EWT) was mixed with approximately 25% by weight of a MMA/VP cross-linked co-polymer as described in Example 1. The resultant paste was shaped in to 10 mm long rods, as described in Example 1. Immediately after moulding, the rods were hydrated in distilled water. After 24 hours' hydration, the sealant composition exhibited a weight increase of approximately 13%. After 48 hours' hydration, the weight increase was approximately 21%, which rose to 31% after 144 hours.

1. A sealant composition, suitable for surgical or dental use, which comprises a sealant and, dispersed therein, one or more particles of a material which is prestressed and/or capable of undergoing expansion or contraction.

2. The composition according to claim 1, wherein the material is capable of undergoing expansion.

3. The composition according to claim 2, wherein the material is capable of undergoing anisotropic expansion.

4. The composition according to claim 1, wherein the material is thermolabile.

5. The composition according to claim 1, wherein the material is prestressed.

6. The composition according to claim 1, wherein the material is hydrophilic.

7. The composition according to claim 6, wherein the material has an equilibrium water uptake of from 10 to 99%.

8. The composition according to claim 1, wherein the material is polymeric.

9. The composition according to claim 8, wherein the material is obtainable by the polymerisation of one or more monomers selected from hydroxyethyl methacrylate, acrylonitrile, methacrylonitrile, N-vinyl-2-pyrrolidinone and methyl methylacrylate.

10. The composition according to claim 8, wherein the material is crosslinked.

11. The composition according to claim 1, wherein the particle size is from 5 μm to 1 mm.

12. The composition according to claim 1, which comprises 2 to 80% by volume of the particles.

13. The composition according to claim 1, wherein the sealant is a curable polymeric resin.

14. The composition according to claim 13, wherein the sealant is obtainable by the polymerisation of one or more monomers selected from epoxy amine, cyanoacrylate or methacrylate monomers.

15. The composition according to claim 1, which further comprises a radio-opaque material.

16. The composition according to claim 15, wherein the one or more particles contain the radio-opaque material.

17. The composition according to claim 15, wherein the radio-opaque material is tungsten carbide, barium sulphate, zirconium oxide or bismuth oxide.

18. The composition according to claim 1, which additionally comprises an active agent.

19. A method for the manufacture of a composition for use as a dental or surgical sealant wherein said method comprises the use of a sealant composition, suitable for surgical or dental use, which comprises a sealant and, dispersed therein, one or more particles of a material which is prestressed and/or capable of undergoing expansion or contraction.
